(19) United States
(12) Patent Application Publication
(10) Pub. No.: US 2007/0004692 A1
(43) Pub. Date: Jan. 4, 2007

(54) Preparations and therapy of intrathecal inflammatory disease

(76) Inventors: Arnd Doerfler, Erlangen (DE); Michael Forsting, Essen (DE); Sophia Goericke, Essen (DE); Ulrich Speck, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/443,440

(22) Filed: May 31, 2006

Related U.S. Application Data
(60) Provisional application No. 60/685,879, filed on Jun. 1, 2005.

(51) Int. Cl. A61K 31/573

(52) U.S. Cl. 514/179

(57) ABSTRACT

A method for prevention and/or treatment of inflammatory/edematous diseases of the central nervous system or increased intracranial pressure comprises intrathecal administration of at least one anti-inflammatory and/or anti-edematous agent.

This application claims the benefit of the filing date of U.S. Provisional Application Ser. No. 60/685,879 filed Jun. 1, 2005.

Inflammation is an important component of many diseases of the central nervous system. It can also worsen the condition after injury, surgery or stroke. Therapy of inflammatory processes of the central nervous system is difficult due to the required transfer of drugs through the blood-brain barrier and systemic and local adverse effects. One of the prominent diseases requiring fast and effective therapy to limit damage to the least possible extent is stroke. We selected stroke as an important example for testing new concepts for treating inflammatory diseases and reaction of the CNS. Ischemic stroke represents the third leading disease in industrial countries frequently causing mortality or disability (Grau et al., 2001). Large unilateral hemispheric infarctions occur in 10-15% of stroke patients and may lead to massive cerebral edema with increased intracranial pressure, clinical deterioration, and death in up to 80% of patients (Hacke et al. 1996; Pullicino et al. 1997). Aggressive therapies, such as thrombolysis, decompressive craniectomy, and hypothermia aim to restore blood flow and reduce postischemic brain edema and have shown to be beneficial in selected patients. Unfortunately, most procedures are invasive, and/or require specific equipment, personal and environment; furthermore, the therapeutic time window may be particularly short in these settings (Brott and Bogousslavsky, 2000).

Various classes of pharmacological neuroprotective agents such as free radical scavengers, glutamate antagonists, immune modulating, anti-inflammatory and neuropharmacological agents have been studied for the treatment of acute ischemic stroke. Despite promising results in experimental settings, only few have reached clinical controlled trials mainly without demonstrating a clinical benefit (Hacke, 2002; Sareen, 2002).

Glucocorticoid steroids are powerful antiinflammatory agents which reach the brain in spite of the blood-brain barrier. They are clinically widely established for numerous indications, primarily act as membrane stabilizers, antioxidants and anti-inflammatory agents (Abraham et al. 2001). Interestingly, there are a few experimental and clinical studies on the effects of glucocorticoids in acute cerebral ischemia available reporting controversial results (Abraham et al., 2001; Adachi et al., 1998; Bertorelli et al., 1998; de Courten-Myers et al., 1994; Koide et al., 1986; Lapchak et al., 2000; Limbourg et al., 2002; Norris and Hachinski, 1986; Patten et al., 1972; Slivka and Murphy, 2001; Tuor et al., 1993). Some studies indicate a beneficial effect in focal cerebral ischemia, however, in every case with major limitations (Bertorelli et al, 1998; de Courten-Myers et al., 1994; Slivka and Murphy, 2001; Limbourg et al., 2002; Tuor et al., 1993). Using a model of permanent cerebral ischemia in rats, Bertorelli et al., 1998 showed dexamethasone (3 mg/kg b.w.) given intraperitoneally 10 minutes after infarction onset to decrease infarction size by up to 50%. In a model of neonatal hypoxia in seven days old rats, Tuor et al., 1993 demonstrated that dexamethasone pre-treatment at 0.5 mg/kg b.w./day for three days may prevent subsequent cerebral damage associated with 3 hours of cerebral hypoxia-ischemia plus unilateral carotid artery ligation. Administration of intravenous methylprednisolone at high doses (30 mg/kg b.w. over 15 minutes, followed by 5.4 mg/kg b.w./h for 23 hours) 30 minutes after onset of cerebral ischemia may significantly reduce infarction volume and cerebral blood flow drop in cats surviving 4 days (de Courten-Myers et al., 1994). In other studies administration of glucocorticoids resulted in no or even detrimental effects on infarction size (Adachi et al., 1998; Koide et al., 1986; Slivka and Murphy, 2001; Sapolsky, 1990). Koide et al., 1986 found that glucocorticoids administered intraperitoneally did not improve (acute pre-treatment and post treatment) but rather even aggravated (chronic pre-treatment) infarction size in transient focal forebrain ischemia in rats. High dose methylprednisolone (105 mg/kg b.w. intraarterially) decreased infarction volume following temporary, but not permanent, focal ischemia in spontaneously hypertensive rats (Slivka and Murphy, 2001).

More than 30 years ago Patten et al., 1972 published a clinical double-blind placebo-controlled study indicating potential benefits of intravenous and intramuscular Dexamethasone in 15 steroid-treated patients versus placebo. The difference was, however not statistically significant. Significance was only reached after subsequent selecting the patients to be evaluated. In a double-blind, randomized, controlled patient trial, high dose dexamethasone given within 48 hours after onset of stroke resulted in no significant difference in overall neurological outcome or death. However, the authors stated that steroid administration might prevent death in patients harboring massive infarcts (Lapchak et al., 2000). These observations reflect the heterogeneous features involved in neurodegenerative and neuroprotective processes.

In all previous studies, corticoids were administered systemically. Clinically, elevation of blood glucose levels by glucocorticoids is reported to be detrimental and is associated with increased short- and long-term mortality in acute cerebral ischemia (Williams et al., 2002).

The negative effects reported are possibly due to the systemic application, leading to an increased blood glucose level.

It would be desirable to overcome the problems with the existing therapies and provide fast acting efficacious inhibition of inflammation, edema and intracranial pressure by simple and less invasive methods which are still beneficial if used several hours after the insult.

Intrathecal corticoid administration was found to bypass these systemic effects and thereby decreases brain edema and intracranial pressure, preserving collaterals. Functionally compromised but viable brain has an increased chance to survive.

Using new preparations of antiinflammatory drugs and a different route of administration we found surprisingly useful effects in severe conditions, e.g., toleration of therapeutic doses of antiinflammatory agents when directly injected in the cerebrospinal fluid, arrival of the drug at the infarcted area, fast and sustained beneficial effects and—very important—effectiveness even if applied at later time after the deleterious event when the patient is more likely to be treated or may reach a hospital.

### DESCRIPTION OF THE INVENTION

Suitable drugs belong to the classes of anti-inflammatory and anti-edematous agents. Examples not limiting the choice of agents are corticosteroids, non-specific and COX-2 selective non-steroidal anti-inflammatory agents, immunesuppressants and anti-hyperplastic agents. Of particular interest are corticosteroids, in particular corticosteroids in their base forms and derivatives thereof, in particular acetal derivatives as well as esters of corticosteroids and derivatives thereof and salts of corticosteroids and derivatives and esters thereof. Salts of corticosteroids exhibit a largely improved water solubility and are therefore commonly used for intravenous injection. Preferred salts according to the present invention are Betamethasone sodium phosphate, Dexamethasone sodium phosphate, Hydrocortisone sodium phosphate, Prednisolone sodium phosphate, Hydrocortisone sodium succinate, and Methylprednisolone sodium succinate.

Corticosteroids in their base forms and acetal derivatives thereof as well as esters thereof exhibit a comparably low water solubility and are generally lipophilic. The use of such corticosteroids is especially preferred.

Preferred esters of glucocorticoids are Triamcinolone diacetate, Hydrocortisone cypionate, Hydrocortisone acetate, Methylprednisolone acetate, Prednisolone acetate, Prednisolone tabulate, Cortisone acetate, Dexamethasone acetate, Betamethasone benzoate, and Betamethasone dipropionate,

Especially preferred corticosteroids in their base forms and acetal derivatives thereof are Betamethasone, Cortisone, Dexamethasone, Fluprednisolone, Hydrocortisone, Meprednisolone, Paramethasone, Prednisolone Triamcinolone, Triamcinolone Hexacetonide, and Triamcinolone acetonide.

Most preferred are Triamcinolone, Triamcinolone acetonide and dexamethasone.

According to the present invention, one or more of the anti-inflammatory and/or antiedematous compounds may be administered intrathecally.

When more than one anti-inflammatory and/or antiedematous compound is administered intrathecally, mixtures of such compounds may be administered or they can be administered separately.

Preferred are lipophilic drugs (partition coefficient between n-butanol and water>0.5, preferentially>10 and more preferentially>100) with low water-solubility, and their derivatives releasing the active compound in vivo. Although prodrugs releasing the active compound following injection may be applied the active agents are preferred. Mixtures of drugs displaying immediate biological activity with e.g. lipophilic drugs or prodrugs acting for a prolonged period of time are useful.

Surprising advantages have been found by choosing the intrathecal route of injection. The invention therefore relates in one embodiment to a method for prevention and/or treatment of inflammatory/edematous diseases of the central nervous system or increased intracranial pressure comprising intrathecal administration of at least one anti-inflammatory and/or anti-edematous agent.

In a preferred embodiment the anti-inflammatory and/or anti-edematous agent is a glucocorticoid. In an especially preferred form the glucocorticoid is in its base form, or is an ester and/or acetal thereof. In particular, the acetal derivative is an acetonide.

As surprisingly low doses of glucocorticoids are necessary to obtain the observed effects, it is possible to administer aqueous solutions of the lipophilic glucocorticoids in their base form or acetonide form or ester form.

Thus, in an especially preferred embodiment, the glucocorticoids in their base form or acetonide form or ester form are administered as an aqueous solution.

It is thus a preferred embodiment of the present invention that aqueous solutions of glucocorticoids in their base form or acetonide derivatives thereof or esters thereof are administered intrathecally for the treatment of inflammatory/edematous diseases of the central nervous system or increased intracranial pressure, in particular stroke. Inventors have surprisingly found that in the animal model, intrathecal administration of the glucocorticoids leads to beneficial effects even 4 hours after induction of focal cerebral ischemia (see example 5). Considering the slower metabolism in humans this translates to an extraordinarily long time window for treatment. This is of major importance as state of the art treatment with t-PA can only be effective when administered within 6 hours after the stroke. Many other drugs and routes of administration must be applied much earlier. Some drugs are only effective if given before the deleterious event occurs. As the time when brain infarction occurs is unpredictable and disease is very often not diagnosed immediately, and as transportation to specialized hospitals is often time-consuming, patients often arrive too late at the hospital to benefit from the treatment with t-PA or a related agent active in the lysis of blood clots (trombolysis).

The compositions and methods of the invention offer much wider treatment windows.

Administration can occur prior to the brain insult, e.g., surgery or stroke, or immediately after, or up to 4 hours or more, or up to 12 hours or more after the insult, or up to 24 hours or even longer thereafter.

In a preferred embodiment, the administration occurs up to 24 hours after stroke, preferably up to 12 hours after stroke.

In another preferred embodiment, the administration to humans occurs between 4 and 24 hours preferably between 6 and 12 hours after stroke.

A further embodiment of the present invention relates to a combination treatment of ischemic stroke, wherein

(a) at least a method of the present invention is practiced and

(b) a clot lysis treatment is performed

The clot lysis treatment is preferably employing t-PA.

Intrathecal administration may be safely and easily performed in patients by lumbar injection but also by the cervical or intracisternal route or by injection into the ventricles. To avoid an increase in pressure due to the injection volume an equal amount of cerebrospinal fluid may be withdrawn through the same needle used for the injection. In spite of the sensitivity of the CNS therapeutic doses can be administered. Intrathecal administration even reduce undesired effects of the therapeutic agents.

Drug doses and frequency of administrations depend on efficacy and tolerance. They will be selected for each drug according to standard pharmacological experiments and clinical dose finding studies. Single injections are preferred. Injection volumes are preferentially chosen between 1 and 30 ml, most preferred are volumes between 5 and 15 ml.

The administered dose of anti-inflammatory and/or anti-edematous agent may vary. Generally, the dose for a glucocorticoid or a derivative thereof is about 0,1 mg to 20 mg, in particular about 1 to 5 mg (e.g. dissolved in 5 to 15 ml). Preferably, the dose is administered in a single injection.

Preparations are those known in pharmacy used for injection, e.g. sterile aqueous solutions containing the excipients to adjust osmolality and pH and to improve the stability of the drug substance in solution or contrast media to allow visualization of the distribution following injection. Furthermore, emulsions, suspensions or liposomal preparations or any other water-miscible fluid preparations may be used. Preferred are novel preparations specifically designed for the purpose, e.g. intrathecal injection and prolonged action after a single administration. Such preparations may contain the drug at concentrations beyond the solubility in water, even more preferred are preparations in which an increased solubility of the active ingredient is achieved while a large proportion (e.g.>50%) of the drug is not enclosed in particles, micelles or other complex supramolecular structures. Solubility of drugs in aqueous solutions may be enhanced by well tolerated organic solvents, or specific solubility enhancers such as cyclodextrins or aromatic compounds (e.g. X-ray contrast media). Admixing the drug to an x-ray contrast agent increases the stability of supersaturated solutions (see example 7) and allows to control injection and distribution of the preparation.

If not stable for a sufficient period of time the drugs may be provided in a small volume of solvent (X) to be added to the usually larger volume of solvent or solution (Y) which improves the properties of the final preparation e.g. in respect of tolerability. Either one or both preparations may be provided in syringes ready for injection or in containers or syringes with two or more chambers separating the components of the final preparation during storage.

Furthermore, therapeutically effective doses of drugs in preferred injection volumes of e.g<than 10 to 30 ml may be achieved by applying 2 or more different substances each of them soluble at a subtherapeutic dose/concentration. Whereas the drugs do not interact in respect of solubility each of them contributes to the efficacy.

The invention in a further embodiment relates to a pharmaceutical preparation containing at least one anti-inflammatory and/or anti-edematous agent in therapeutic, non toxic dosage, in sterile, pyrogen-free aqueous solution, for intrathecal administration.

In a preferred embodiment, the anti-inflammatory and/or anti-edematous agent is a glucocorticoid in its base form or acetonide form or ester form, preferably base form or acetonide form.

In a further embodiment, the invention relates to a liquid pharmaceutical preparation containing at least one anti-inflammatory and/or anti-edematous agent in therapeutic, non toxic dosage at a concentration not soluble in water, wherein a therapeutically effective proportion of the drug is not contained in supramolecular structures.

In a further embodiment, the invention relates to a liquid pharmaceutical preparation containing at least one anti-inflammatory and/or anti-edematous agent in therapeutic, non toxic dosage at a concentration not soluble in water, and a X-ray contrast agent.

Such X-Ray contrast agent surprisingly lead to enhanced stability of pharmaceutical preparations according to the present invention (see example 7). In an especially preferred embodiment the X-Ray contrast agent is Ultravist® or Isovist®.

Preferably, the pharmaceutical preparation is an aqueous solution.

In a further embodiment, the invention relates to a method for the treatment of a brain infarct, trauma of the central nervous system and/or conditions following central nervous system surgery, comprising administering intrathecally one of the compositions and preparations of the present invention.

In a further embodiment, the invention relates to a kit consisting of
(a) at least one anti-inflammatory and/or anti-edematous agent in therapeutic, non toxic dosage, preferably in sterile pyrogen-free stable solution (A) and
(b) at least one pharmaceutical preparation

Both components can be mixed before administration. The pharmaceutical preparation to be used in step b) may be an X-ray contrast agent or another pharmaceutically acceptable aqueous or water miscible non-aqueous solution.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

The entire disclosures of all applications, patents and publications, cited herein are incorporated by reference herein.

### FIGURE LEGEND

FIG. 1: Mean glucose values of Example 6 in course of time: preoperatively, immediately prior to, at 2 hours and at 24 hours after intrathecal Triamcinolone acetonide-application; control=saline

### EXAMPLES

### Example 1

Microcrystal suspension of Triamcinolone acetonide 40 mg/ml (Bristol-Myers Squibb) is diluted with sterile saline 1+249

### Example 2

Microcrystal suspension of Triamcinolone acetonide (Bristol-Myers Squibb) is diluted with sterile saline 1 ml+249 ml 4.6 ml Gadolinium DTPA, 0.5 M, Magnevist®, Schering AG, Berlin, Germany are added

### Example 3

40 mg Triamcinolone acetonide (Sigma-Aldrich, St. Louis) is dissolved in 4 ml ethanol. 0.4 ml of this preparation is added to 25 ml sterile saline. The solution is filtered to eliminate pyrogens.

### Example 4

Animal Preparation

Anaesthesia was induced by inhalative isoflurane and maintained by intramuscular injection of a mixture of ketamine 10% and xylazine 2% (7:3) at a dose of 0.1 ml/100 g bodyweight. Animals breathed spontaneously. The right femoral artery was catheterised to monitor the mean arterial blood pressure, heart rate, PaO₂ and PaCO₂ during animal preparation. Body temperature was controlled rectally and maintained normothermic at 37.5±0.5° C. by applying external heat as needed using a heating pad.

Focal cerebral ischemia was introduced using an intraluminal suture occlusion model of the middle cerebral artery (MCA)) (Longa et al, 1989). Briefly, the external carotid artery was ligated, the internal carotid artery (ICA) was isolated and the pterygopalatine artery was ligated. A 4-0 monofilament nylon suture, whose tip was coated with silicone, was introduced transvascularily via an arteriotomy into the common carotid artery and gently advanced through the ICA into the origin of the anterior cerebral artery, thus occluding the origin of the MCA.

Study

Animals were randomly assigned to two groups, 15 animals each. The treatment group received Triamcinolone acetonide 0.012 mg/kg intrathecally according to example 2, the control group received equivolumetric saline.

To confirm successful MCA occlusion diffusion weighted MRI was performed.

Intrathecal drug administration was performed 30 minutes after MCA occlusion as a single middle suboccipital injection (volume of either preparation according to example (2) or saline of 0.075 ml/kg b.w.) into the cisterna magna performed slowly using a 23 G needle with the animals still under anaesthesia.

Twenty-four hours after MCAO animals were sacrificed, brains rapidly removed and 2 mm coronal brain slices were incubated for 30 minutes in a 4% solution of 2,3,5triphenyl-tetrazolium-chloride (TTC) at 37° C. and fixed by immersion in 4.5% buffered formalin solution. Brain slices were photographed from both sides, the area of infarction was quantified using IMAGE 1.41 (NIH, Bethesda Md., USA). On each slice the infarction volume (non-stained area) was marked and calculated by an observer blinded to the animals' experimental group. Both sides of each brain slice were measured separately, and mean values were calculated. Since total brain volume in the different treatment groups varied due to the different body weight of animals at baseline, we calculated relative infarction volumes expressed as percent of the total brain volume of the right hemisphere. The average percent infarction volume was calculated for each group.

Results:

T1-weighted MRI confirmed successful intrathecal administration and diffusion weighted imaging successful MCA occlusion in all evaluated animals. Compared to controls, infarction volume was significantly reduced in animals treated with Triamcinolone acetonide intrathecal treatment (13.4±6.5% vs. 20.0±8.0%, p=0.02), representing a 33% reduction in infarction size of compared to controls.

### Example 5

The study was performed using the same animal model as in example 4. Intrathecal injection of Triamcinolone acetonide, 0.012 mg/kg, according to example 3 was performed at various points in time up to 4 h following middle cerebral artery occlusion. Single dose at the time indicated. The results are shown in Table 1.

The results indicate excellent efficacy of intrathecal drug even 4 hours after infarction. Considering the slower metabolism in humans this translates to an extraordinarily long time window for treatment.

### Example 6

In the animals used in ‘Example 5’ additionally blood glucose was measured with glucose oxidase reagent strips (Accu-Chek® Sensor, Roche Diagnostik GmbH, Mannheim, Germany) using a drop of venous blood preoperatively, immediately prior to, at 2 hours and at 24 hours after Triamcinolone acetonide administration.

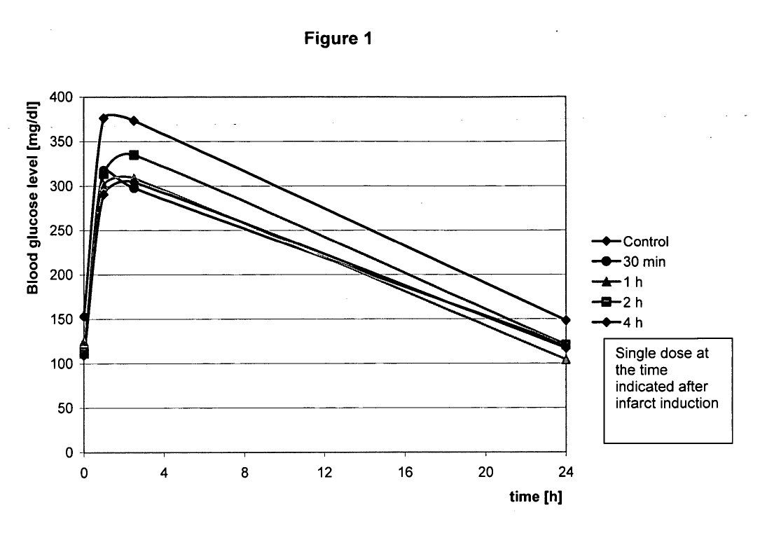

Blood glucose increases significantly following surgery and infarction. Intrathecal corticoid does not cause any additional effect. The results are shown in FIG. 1.

### Example 7

20 mg Triamcinolone acetonide or dexamethasone was dissolved in 1.0 ml absolute ethanol. 0.16 ml was added to 10 ml of saline or the x-ray contrast agents Ultravist™ (radioopaque ingredient iopromide) or Isovist™ (radioopaque ingredient iotrolan) both Schering AG, Berlin, Germany. Whereas the solubility at room temperature in water is about 20 μg/ml for Triamcinolone acetonide and about 85 μg/ml for dexamethasone by adding the drug dissolved in a small volume of ethanol solutions of 3.2 mg/10 ml were achieved. Contrary to the solutions in saline the solutions in Ultravist™ remained physically stable for at least 24 hours; Isovist™ delayed precipitation as well but not to the same extent. According to the animal experiments a dose of about 3 mg/10 ml (10 ml per dose) is expected to provide a therapeutic effect in human patients. The results are shown in Table 2.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

TABLE 1

| | | Drug administration after MCAO | | | |
|---|---|---|---|---|---|
| | Control (saline) | 30 Min | 1 h | 2 h | 4 h |
| Mean | 18.08 | 12.86* | 9.68** | 10.57* | 9.05** |
| SD | 2.80 | 5.39 | 4.53 | 6.21 | 5.15 |
| n | 15 | 15 | 15 | 15 | 15 |

Relative infarction volumes expressed as percent of the total brain volume of the right hemisphere (*p < 0.05, **p < 0.01 versus control)

TABLE 2
Physical stability of aqueous corticosteroid solutions

| | Triamcinolone Acetonide | | | Dexamethasone | | |
|---|---|---|---|---|---|---|
| Time after | | Ultravist ™ | | | Ultravist ™ | Isovist ™ |
| mixing | Saline | 370 | Isovist ™ 300 | Saline | 300 | 300 |
| 0 | clear (?) | clear | clear | clear | clear | clear |
| 2 h | slightly | clear | clear | clear | clear | clear |
| | turbid | | | | | |
| 48 h | crystals on | clear | fine particles | crystals on | clear | clear |
| | glass | | | the wall | | |

1) A method for prevention and/or treatment of inflammatory/edematous diseases of the central nervous system or increased intracranial pressure comprising intrathecal administration of at least one anti-inflammatory and/or anti-edematous agent.

2) A method according to claim 1, wherein the inflammatory/edematous diseases of the central nervous system is stroke, in particular ischemic stroke.

3) A method according to claim 1, wherein the anti-inflammatory and/or anti-edematous agent is a glucocorticoid.

4) A method according to claim 2, wherein the glucocorticoid is in its base form.

5) A method according to claim 3, wherein the glucocorticoid is selected from Betamethasone, Cortisone, Dexamethasone, Fluprednisolone, Hydrocortisone, Meprednisolone, Paramethasone, Prednisolone and/or Triamcinolone.

6) A method according to claim 1, wherein the administered volume is about 1 ml to about 30 ml, preferably between about 5 ml and about 10 ml.

7) A method according to claim 1, wherein the administered dose of anti-inflammatory and/or anti-edematous agent is 0,1 mg to 20 mg, in particular 1 to 5 mg.

8) A method according to claim 1, wherein the administration is performed up to 24 hours after stroke

9) A method according to claim 8, wherein the administration is performed between 4 and 24 hours after stroke.

10) A pharmaceutical preparation containing at least one anti-inflammatory and/or anti-edematous agent in therapeutic, non toxic dosage, in sterile, pyrogen-free aqueous solution, for intrathecal administration.

11) A pharmaceutical preparation according to claim 10, wherein the anti-inflammatory and/or anti-edematous agent is a glucocorticoid in its base form or acetonide form or ester form, preferably base form or acetonide form.

12) A liquid pharmaceutical preparation containing at least one anti-inflammatory and/or anti-edematous agent in therapeutic, non toxic dosage at a concentration not soluble in water, wherein a therapeutically effective proportion of the drug is not contained in supramolecular structures.

13) A liquid pharmaceutical preparation containing at least one anti-inflammatory and/or anti-edematous agent in therapeutic, non toxic dosage at a concentration not soluble in water, and a X-ray contrast agent.

14) A liquid preparation according to claim 13, wherein the pharmaceutical preparation is an aqueous solution.

15) A method for the treatment of a brain infarct, trauma of the central nervous system and/or conditions following central nervous system surgery, comprising administering intrathecally a preparation of claim 10.

16) A kit consisting of at least one anti-inflammatory and/or anti-edematous agent in therapeutic, non toxic dosage, preferably in sterile pyrogen-free stable solution (A) and at least one pharmaceutical preparation
